(19) United States
(12) Patent Application Publication
(10) Pub. No.: US 2025/0339370 A1
(43) Pub. Date: Nov. 6, 2025

(54) PERFORMANCE-ENHANCING EXCIPIENTS AND METHODS OF REDUCING VISCOSITY AND INCREASING STABILITY OF BIOLOGIC FORMULATIONS

(71) Applicant: Avantor Performance Materials, LLC, Radnor, PA (US)

(72) Inventors: Arvind Srivastava, Stewartsville, NJ (US); Nandkumar Deorkar, Cedar Knolls, NJ (US); Lori Fortin, Bethlehem, PA (US); Courtney O'Dell, Denville, NJ (US); Evon Bolessa, Edison, NJ (US)

(73) Assignee: Avantor Performance Materials, LLC, Radnor, PA (US)

(21) Appl. No.: 18/285,918

(22) PCT Filed: Apr. 8, 2022
(86) PCT No.: PCT/US2022/023966

§ 371 Date: Oct. 6, 2023

Related U.S. Application Data
(60) Provisional application No. 63/173,055, filed on Apr. 9, 2021.

(51) Int. Cl. A61K 9/08; A61K 47/02; A61K 47/18; A61K 47/22; A61K 47/26; C07K 16/00

(52) U.S. Cl. CPC A61K 9/08; A61K 47/02; A61K 47/183; A61K 47/22; A61K 47/26; C07K 16/00

(57) ABSTRACT

The present invention relates to the viscosity reduction and stability enhancement of biotherapeutics in biomanufacturing and formulation. The method of viscosity reduction and stability enhancement comprises combining a biotherapeutic with a performance-enhancing excipient chosen from bis acetyl arginine, bis acetyl lysine, bis acetyl histidine, bis acetyl serine, bis acetyl proline, bis acetyl tryptophan, propionyl arginine, propionyl lysine, propionyl histidine, propionyl serine, propionyl proline, propionyl tryptophan, and mixtures thereof.

### FIELD OF THE INVENTION

The present invention relates to performance-enhancing excipients which minimize solution viscosity and physical and chemical degradation of biotherapeutics by, for example, inhibiting protein-protein interactions and post translational modifications. Additionally, this invention provides methods of using performance-enhancing excipients for bioprocessing and for biologic formulations comprising protein therapeutics, peptides, antibodies, antibody drug conjugates (ADC), gene therapy, cell therapy, nucleic acids etc.

### BACKGROUND OF THE INVENTION

Biologics manufacturing (bioprocessing) that utilizes recombinant technology is a complex process. Typical bioprocessing steps include: (i) upstream processing, where product is manufactured; (ii) downstream processing, where product is purified and (iii) formulation/fill and finish, where product is formulated to maintain desired product quality attributes throughout the shelf-life. Biologics can undergo various physical/chemical degradations during manufacturing, storage, shipping, and handling, which reduce therapeutic effects and raise safety concerns. Examples of biologic products include protein therapeutics, peptides, antibodies, antibody drug conjugates (ADC), nucleic acids, and gene and cell therapy.

Biologics are frequently formulated in liquid solutions, particularly for parenteral administration. There are two main routes of administration for parenteral products: i) intravenous administration and ii) subcutaneous administration. Stability loss resulting from stresses, such as those caused by temperature excursions, shear force, freeze/thaw, light exposure, oxidation, etc., are common in both intravenous and subcutaneous formulations. However, subcutaneous administration poses additional challenges due to often large doses and a small delivery volume limitation of 1-2 ml. Typically, subcutaneous formulations in delivery volumes greater than 1-2 ml are not well tolerated by the patient. In such cases, highly concentrated product formulations may be desirable to meet the limited dose volume. The high dose and small volume requirements for subcutaneous administration means that the product concentration reaches upwards of 100 mg/ml or more. Highly concentrated formulations can pose many challenges to the manufacturability, analytical testing, and administration of protein therapeutics. One challenge posed by highly concentrated protein formulations is increased viscosity. High viscosity biologics are difficult to handle during manufacturing, e.g., they slow down tangential flow filtration (TFF) and aseptic filtration processes and increase the product loss during processing. High viscosity formulations are also difficult to draw into a syringe and inject, making administration to the patient difficult and unpleasant. The other challenge with high concentration formulations is stability. High concentration biologic solutions often experience “crowded” environments in solution, forming a network of reversible protein-protein interactions, or self-associations. Drug manufacturers typically use amino acids such as arginine and histidine, and salts such as sodium chloride to minimize the solution viscosity in a high concentration formulation. However, often times these additives reduce viscosity at the cost of stability, where viscosity and stability both decrease with the addition of additives. Therefore, there is a need in the industry for compounds that are efficient in reducing viscosity of biologic formulations (e.g., highly concentrated protein formulations) and are effective at stabilizing products in a wide product concentration range and across many therapeutic modalities. The concentration range suitability will offer the manufacturer the ability to store drug substances at higher concentrations and formulate drug products at either low or high concentration as business demands.

### SUMMARY OF THE INVENTION

In one aspect, the performance-enhancing excipients comprising compounds shown in FIG. 1 reduce the viscosity of the high concentration biologics and enhance their physical and chemical stabilities by reducing protein-protein interactions and preventing deamidation of asparagine.

In another aspect, the performance-enhancing excipients comprising compounds shown in FIG. 1 are suitable for use in bioprocessing, e.g., they minimize physical and chemical degradation of biologics during manufacturing, and reduce the solution viscosity that eases TFF, aseptic filtration and bulk/drug product filling operation.

In one aspect, the present invention relates to biologic formulations (e.g., protein therapeutics, peptides, antibodies, antibody drug conjugates (ADC), gene therapy, cell therapy, nucleic acids etc.) which comprise performance-enhancing excipients, and, optionally, surfactant carbohydrates, salts and amino acids. In one embodiment, the performance-enhancing excipients minimize solution viscosity and physical and chemical degradation of proteins by inhibiting protein-protein interactions and post-translational modifications. The performance-enhancing excipients contain functional groups that interact with proteins by hydrophobic interactions, ionic interaction, and hydrogen bonding, resulting in viscosity reduction and physical and chemical stability enhancement. In one embodiment, the excipients are chemically synthesized, for example, by derivatization of amino acids.

In one embodiment, the present invention provides a method for reducing viscosity and/or increasing stability of a biologic formulation comprising: combining the biologic formulation with a performance-enhancing excipient selected from the group consisting of bis acetyl arginine, bis acetyl lysine, bis acetyl histidine, bis acetyl serine, bis acetyl proline, bis acetyl tryptophan, propionyl arginine, propionyl lysine, propionyl histidine, propionyl serine, propionyl proline, propionyl tryptophan, and mixtures thereof. The biologic formulation can comprise a therapeutic protein at a concentration of about 1 mg/ml to about 500 mg/ml, to provide an enhanced formulation. In one embodiment, the biologic formulation further comprises an additional excipient, wherein the performance-enhancing excipient is in a concentration of about 5 mM to about 1000 mM.

In one embodiment, the performance-enhancing excipient is bis acetyl arginine. In one embodiment, the performance-enhancing excipient is at least one of the following: bis acetyl lysine, bis acetyl histidine, bis acetyl serine, bis acetyl proline, bis acetyl tryptophan, propionyl arginine, propionyl lysine, propionyl histidine, propionyl serine, propionyl proline, propionyl tryptophan. In one embodiment, the performance-enhancing excipient is a mixture of propionyl serine and bis acetyl lysine in the ratio of about 10 wt. %:90 wt. % to about 90 wt. %:10 wt. %.

In one embodiment, the viscosity of the biologic formulation is reduced by at least about 10% to about 80%. In one embodiment, the enhanced formulation has superior stability compared to buffer control. In one embodiment, the enhanced formulation has higher monomer compared to buffer control upon exposure to stressed temperature conditions. In one embodiment, the enhanced formulation has lower aggregate compared to buffer control upon exposure to stressed temperature conditions. In one embodiment, the enhanced formulation has lowered degradant compared to buffer control upon exposure to stressed temperature conditions. In one embodiment, the enhanced formulation has a lower change in percent acidic peak group (APG) compared to buffer control upon exposure to stressed temperature conditions.

In one embodiment, the enhanced formulation has a pH between about 4.0 to about 9.0. In one embodiment, the enhanced formulation is in the form of a lyophilized powder, wherein the at least one performance-enhancing excipient is present at a weight:weight concentration effective to reduce viscosity upon reconstitution with a diluent. In one embodiment, the performance-enhancing excipient is present at a concentration of between about 5 mM to about 1000 mM, and the therapeutic protein is present at a concentration of about 1 mg/ml to about 500 mg/ml. In one embodiment, the biologic formulation is at least one of protein therapeutics, peptides, antibodies, antibody drug conjugates (ADC), nucleic acids, gene therapy and cell therapy.

### BRIEF DESCRIPTION OF THE DRAWINGS

FIG. 1: is a chemical structure of the performance-enhancing excipients of the present invention. Each excipient varies in its R¹, R² and R³ groups.

FIG. 2: is a typical size exclusion chromatogram (SEC-HPLC) for a mAb. The monomer, aggregate and degradant peaks are identified in the Figure.

FIG. 3: is a typical ion exchange chromatogram (IEC-HPLC) for a mAb. The acidic peak group (APG) is identified in the Figure.

FIG. 4: is a graph showing the viscosity of mAb formulations in Table 2 at 25° C.

FIG. 5: is a graph showing the viscosity of mAb formulations in Table 3 at 25° C.

FIG. 6: is a graph showing the propionyl serine concentration dependent viscosity reduction of mAb at 250 mg/ml in 10 mM phosphate pH 8.0 buffer. The viscosity measurement was done at 25° C.

FIG. 7: is a graph showing the percent monomer of mAb formulations in Table 4 at initial and following 1 and 2 weeks of storage at 50° C. Percent monomer was determined using SEC-HPLC.

FIG. 8: is a graph showing the percent aggregate of mAb formulations in Table 4 at initial and following 1 and 2 weeks of storage at 50° C. Percent monomer was determined using SEC-HPLC.

FIG. 9: is a graph showing the percent degradant in mAb formulations in Table 4 at initial and following 1 and 2 weeks of storage at 50° C. Percent monomer was determined using SEC-HPLC.

FIG. 10: is a graph showing the percent acidic peak group (APG) in mAb formulations in Table 4 at initial and following 1 and 2 weeks of storage at 50° C. Percent APG was determined using IEC-HPLC.

FIG. 11: is a graph showing the percent monomer in mAb formulations in Table 4 at initial and following 2 and 4 weeks of storage at 40° C. Percent monomer was determined using SEC-HPLC.

FIG. 12: is a graph showing the percent aggregate in mAb formulations in Table 4 at initial and following 2 and 4 weeks of storage at 40° C. Percent aggregate was determined using SEC-HPLC.

FIG. 13: is a graph showing the percent degradant in mAb formulations in Table 4 at initial and following 2 and 4 weeks of storage at 40° C. Percent degradant was determined using SEC-HPLC.

FIG. 14: is a graph showing the percent acidic peak group (APG) in mAb formulations in Table 4 at initial and following 2 and 4 weeks of storage at 40° C. Percent APG was determined using IEC-HPLC.

FIG. 15: is a graph showing the percent monomer of mAb formulations in Table 5 at initial and following 1 and 2 weeks of storage at 50° C. Percent monomer was determined using SEC-HPLC.

FIG. 16: is a graph showing the percent aggregate of mAb formulations in Table 5 at initial and following 1 and 2 weeks of storage at 50° C. Percent aggregate was determined using SEC-HPLC.

FIG. 17: is a graph showing the percent degradant of mAb formulations in Table 5 at initial and following 1 and 2 weeks of storage at 50° C. Percent degradant was determined using SEC-HPLC.

FIG. 18: is a graph showing the percent acidic peak (APG) group of mAb formulations in Table 5 at initial and following 1 and 2 weeks of storage at 50° C. Percent APG was determined using SEC-HPLC.

FIG. 19: is a graph showing the percent monomer of mAb formulations in Table 5 at initial and following 4 and 8 weeks of storage at 40° C. Percent monomer was determined using SEC-HPLC.

FIG. 20: is a graph showing the percent aggregate of mAb formulations in Table 5 at initial and following 4 and 8 weeks of storage at 40° C. Percent aggregate was determined using SEC-HPLC.

FIG. 21: is a graph showing percent degradant of mAb formulations in Table 5 at initial and following 4 and 8 weeks of storage at 40° C. Percent degradant was determined using SEC-HPLC.

FIG. 22: is a graph showing the percent acidic peak group (APG) of mAb formulations in Table 5 at initial and following 4 and 8 weeks of storage at 40° C. Percent APG was determined using IEC-HPLC.

FIG. 23: Percent deamidation of mAb formulations in Table 7. Percent deamidation was determined using mass spectroscopy.

FIG. 24: is a graph showing mutual diffusion (Dₘ) reported as a function of protein concentrations. Measurements were carried out using a Zetasizer Nano ZS series instrument at 25° C.

### DETAILED DESCRIPTION OF THE INVENTION

In one aspect, the invention relates to performance-enhancing excipients that minimize solution viscosity, and physical and chemical degradation of biologic formulations, and improve the physical and chemical stabilities of the formulations. For example, the performance-enhancing excipients reduce protein-protein interactions (PPI) and post translational modifications. Examples of chemical degradation include oxidation, deamidation, hydrolysis, disulfide exchange, β-elimination etc. Examples of physical stability includes unfolding, aggregation, degradation, precipitation, particulate formation, surface adsorption etc.

The excipients of the present invention are suitable for use with a variety of biologic formulations such as, for example, drug product modalities, bio-therapeutics, protein therapeutics, peptides, antibodies, antibody drug conjugates (ADC), nucleic acids, gene therapy and cell therapy. Without wanting to be limited by a mechanism of action, it is believed that the mechanism of viscosity increases and degradation pathways are the same across these modalities.

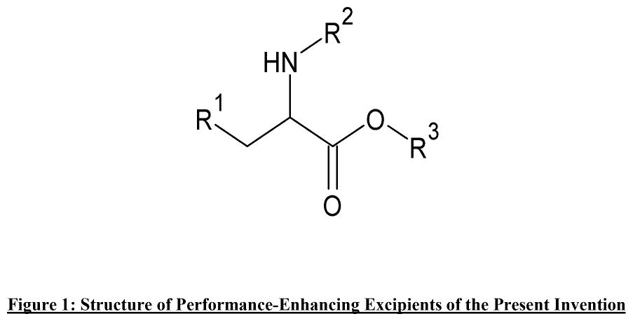

Examples of the performance-enhancing excipients of the present invention include the compounds shown below (and in FIG. 1) and listed in Table 1.

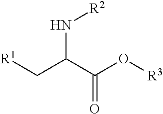

wherein,
R¹═OH—, OCOCH₃, OCOC₂H₅, OCOC₃H₇, OCOC₄H₉, OCOC₅H₁₁, OCOC₆H₁₃, CH₃CONHCH₂CH₂CH₂, C₂H₅CONHCH₂CH₂CH₂, C₃H₇CONHCH₂CH₂CH₂, C₄H₉CONHCH₂CH₂CH₂, C₅H₁₁CONHCH₂CH₂CH₂, C₆H₁₃CONHCH₂CH₂CH₂, SH, SCOCH₃, SCOC₂H₅, SCOC₃H₇, SCOC₄H₉, SCOC₅H₁₁, SCOC₆H₁₃, Indolyl, Indolyl(NCOCH₃), Indolyl(NCOC₂H₅), Indolyl(NCOC₃H₇), Indolyl(NCOC₄H₉), Indolyl(NCOC₅H₁₁), Indolyl(NCOC₆H₃), (OH)(CH₃), (CH₃)(OCOCH₃), (CH₃)(OCOC₂H₅), (CH₃)(OCOC₃H₇), (CH₃)(OCOC₄H₉), (CH₃)(OCOC₅H₁₁), (CH₃)(OCOC₆H₁₃), PhOH, PhOCOCH₃, PhOCOC₂H₅, PhOCOC₃H₇, PhOCOC₄Ho, PhOCOC₅H₁₁, PhOCOC₆H₁₃, CONH₂, CONHCH₃, CONHC₂H₅, CONHC₃H₇, CONHC₄H₉, CONH₂C₅H₁₁, CONH₂C₆H₁₃, CH₂CONH₂, CH₂CONHCH₃, CH₂CONHC₂H₅, CH₂CONHC₃H₇, CH₂CONHC₄H₉, CH₂CONHC₅H₁₁. CH₂CONHC₆H₁₃, CH₂CH₂NHC(NH)NH₂, CH₂CH₂NHC(NH)NHC(O)CH₃, CH₂CH₂NHC(NH)NHC(O)C₂H₅, CH₂CH₂NHC(NH)NHC(O)C₃H₇, CH₂CH₂NHC(NH)NHC(O)C₄H₉, CH₂CH₂NHC(NH)NHC(O)C₅Hu, CH₂CH₂NHC(NH)NHC(O)C₆H₁₃, Imidazolyl, Imidazolyl(NCOCH₃), Imidazolyl(NCOC₂H₅), Imnidazolyl(NCOC₃H₇), Imidazolyl(NCOC₄H₉) Imidazolyl(NCOC₅H₁₁), Imidazolyl(NCOC₆H₁₃), C(O)OH, C(O)OCH₃, C(O)OC₂H₅, C(O)OC₃H₇, C(O)OC₄H₈, C(O)OC₅H₁₁, C(O)OC₆H₁₃, CH₂C(O)OH, CH₂C(O)OCH₃, CH₂C(O)OC₂H₅, CH₂C(O)OC₃H₇, CH₂C(O)OC₄H₈, CH₂C(O)OC₅H₁₁, CH₂C(O)OC₆H₁₃,
R²=H, C(O)CH₃, C(O)C₂H₅, C(O)C₃H₇, C(O)C₄H₉, C(O)C₅H₁₁, C(O)C₆H₁₃
R³=H, CH₃, C₂H₅, C₃H₇, C₄H₉, C₅H₁₁, C₆H₁₃

TABLE 1
Examples of Performance-Enhancing Excipients
of the Present Invention¹:

| Deriv- | |
|---|---|
| atives | Examples |
| Bis acetyl | bis acetyl arginine, bis acetyl lysine, bis acetyl histidine, bis |
| | acetyl serine, bis acetyl proline, bis acetyl tryptophan |
| Propionyl | propionyl arginine, propionyl lysine, propionyl histidine, |
| | propionyl serine, propionyl proline and propionyl tryptophan |
| Butanoyl | butanoyl arginine, butanoyl lysine, butanoyl histidine, |
| | butanoyl serine, butanoyl proline and butanoyl tryptophan |
| Pentanoyl | pentanoyl arginine, pentanoyl lysine, pentanoyl histidine, |
| | pentanoyl serine, pentanoyl proline and pentanoyl tryptophan |
| Hexanoyl | hexanoyl arginine, hexanoyl lysine, hexanoyl histidine, |
| | hexanoyl serine, hexanoyl proline and hexanoyl tryptophan |

¹Both d and l forms of the amino acids are included in this example (e.g. n-propionyl-d serine, n-propionyl-l serine)

In one aspect, the present invention relates to enhanced biologic formulations, for example, protein therapeutics, peptides, antibodies, antibody drug conjugates (ADC), gene therapy, cell therapy, nucleic acids etc., comprising a performance-enhancing excipient of the present invention, and, optionally, a surfactant carbohydrate, salts, and/or amino acids.

In some embodiments, the enhanced biologic formulation is a solution formulation. In such embodiments, at least one of the performance-enhancing excipients is included in a formulation at a concentration range of about 5 mM to about 1000 mM.

In some embodiments, the enhanced biologic formulation is in the form of a lyophilized powder. In such embodiments, at least one of the performance-enhancing excipients is included in a formulation at a weight:weight concentration effective to improve stability and reduce viscosity upon reconstitution with a diluent. The ratio of biologic (e.g., protein) to excipients may vary from about 1:10 (weight:weight) to about 10:1 (weight:weight).

In one embodiment, the present invention provides methods of reducing the viscosity and/or improving stability of biologic formulations. The methods comprise combining a biologic formulation with at least one performance-enhancing excipient of the present invention (e.g., listed in Table 1) to form an “enhanced biologic formulation” (e.g., enhanced protein formulation). In some embodiments, the enhanced biologic formulation further comprises at least one additional excipient.

In one embodiment, a method for reducing the viscosity of a biologic formulation (e.g., liquid pharmaceutical formulation) is provided. The method comprises combining a biologic formulation at a concentration of at least about 1 mg/ml to about 500 mg/ml with at least one performance-enhancing excipient selected from Table 1 to form an enhanced biologic formulation. In a further embodiment, an additional excipient is included which is different from those in Table 1. In these embodiments, the concentration of the performance-enhancing excipient(s) is from about 5 mM to about 1000 mM; and the pH of the formulation is from about pH 4.0 to about pH 9.0. The change in viscosity can vary subject to protein concentration, choice of performance-excipient(s) and their concentrations, solution pH and other formulation components. For example, the viscosity of a formulation can be reduced by at least about 10%, by at least about 30%, by at least about 50%, by at least about 70%, or by at least about 80%.

As used herein, “viscosity” is defined as a fluid's resistance to flow and may be measured in units of centipoise (cP) or milliPascal-second, at a given shear rate. Viscosity may be measured by using a viscometer, e.g., Brookfield Engineering Dial Reading Viscometer, model LVT, and AR-G2, TA instruments. Viscosity may be measured using any other method and in any other units known in the art (e.g., absolute, kinematic, or dynamic viscosity), understanding that it is the percent reduction in viscosity afforded by use of the excipients described by the invention that is important. Regardless of the method used to determine viscosity, the percent reduction in viscosity in an enhanced biologic formulation (e.g., protein formulation) versus a control formulation (i.e., formulations without the excipients of the present invention) will remain approximately the same at a given shear rate.

In one embodiment, a method for stabilization of a biologic formulation (e.g., liquid pharmaceutical formulation) is provided. The method comprises combining a biologic formulation at a concentration of at least about 1 mg/ml to about 500 mg/ml with at least one performance-enhancing excipient from Table 1 to form an enhanced biologic formulation. In this embodiment, the concentration of the performance-enhancing excipient is from about 5 mM to about 1000 mM, and the pH of the formulation is from about pH 4.0 to about pH 9.0. In a further embodiment, an additional excipient is included, wherein such additional excipient is different from those listed in the Table 1.

Stabilization refers to the prevention of change in the quality attributes of a biologic formulation (e.g., therapeutic protein) upon exposure to stress conditions such as temperature, freeze/thaw, shear, light, low/high pH, oxygen and metal impurities etc. In one embodiment, the change in quality attributes refers to change in percentage of monomeric species, aggregate (also referred to as high molecular weight species (HMWS)) and degradant species (also referred to as a low molecular weight species (LMWS)). In another embodiment, the change in quality attributes refers to change in charge variance. An example of a change in charge variance are acidic peak group (APG), basic peak group (BPG) or neutral peak group (NPG). In another embodiment, the change in quality attributes refers to change in functional activities. Examples of functional activities are in-vitro activities, in-vivo activities, binding activities, cell-based activities etc. In another embodiment, the change in quality attributes refers to change in visual appearance and particulate matter in the solution. Examples of changes in visual appearance are change in solution color, sub-visible and visible particulates and/or product precipitation. In another embodiment, the change in quality attributes refers to post translational modifications. Examples of post translational modifications are oxidation, deamidation, isomerization, hydrolysis, disulfide exchange, and β-elimination.

Stability can be assessed in many ways, including monitoring conformational change over a range of temperatures (thermo-stability) and/or time periods (shelf-life) and/or after exposure to stressful handling situations e.g., physical shaking, freeze/thaw and exposure to light. Stability of formulations containing varying concentrations of formulation components can be measured using a variety of methods. For example, the amount of protein aggregation can be measured by visual observation of turbidity, by measuring absorbance at a specific wavelength, by HPLC size exclusion chromatography (in which aggregates of a protein will elute in different fractions compared to the protein in its native active state), or other chromatographic methods. Other methods of measuring conformational change can be used, including using differential scanning calorimetry (DSC) or differential scanning fluorimetry (DSF) to determine the temperature of denaturation, or circular dichroism (CD), which measures the molar ellipticity of the protein. Fluorescence can also be used to analyze conformation. Fluorescence encompasses the release or absorption of energy in the form of light or heat, and changes in the polar properties of light. Fluorescence emission can be intrinsic to a protein or can be due to a fluorescence reporter molecule. For example, ANS is a fluorescent probe that binds to the hydrophobic pockets of partially unfolded proteins. As the concentration of unfolded protein increases, the number of hydrophobic pockets increases and subsequently the concentration of ANS that can bind increases. This increase in ANS binding can be monitored by detection of the fluorescence signal of a protein sample. The change in charge variance can be measured by Ion Exchange Chromatography (IEC-HPLC) where species are separated based on their Isoelectric point (pl). The change in post-translational modifications such as oxidation and deamidation can be measured by LC-MS/MS or reverse-phase HPLC (RP-HPLC). Other methods for measuring stability can be used and are well known to persons of skilled in the art.

Without wanting to be bound to a mechanism of action, it is believed that the performance-enhancing excipients have structural properties that enable them to interact with biologic molecules by hydrophobic, hydrogen bonding and/or ionic interaction mechanisms, resulting in a reduction of protein-protein interaction and protecting susceptible amino acids from post translational modifications.

Examples of additional excipients or stabilizers, that are different from those listed in Table 1, include sugars (e.g., sucrose, glucose, trehalose, fructose, xylose, mannitose, fucose), polyols (e.g., glycerol, mannitol, sorbitol, glycol, inositol), amino acids or amino acid derivative (e.g., arginine, proline, histidine, lysine, glycine, methionine, etc.) or surfactant carbohydrates (e.g., polysorbate, including polysorbate 20, or polysorbate 80, or poloxamer, including poloxamer 188, TPGS (d-alpha tocopheryl polyethylene glycol 1000 succinate)). The concentration of a surfactant may range from about 0.001% to about 20.0%. The concentration of the other additional excipients may vary from about 5 mM to about 2000 mM.

In some embodiments, the enhanced biologic formulation may also include preservatives such as, for example, benzyl alcohol, phenol, m-cresol, chlorobutanol and benzethonium chloride at concentrations ranging from about 0.1% to about 2%.

In some embodiments, the enhanced biologic formulation may also include pharmaceutically acceptable salts and buffers. Examples of pharmaceutically acceptable buffers include phosphate (e.g., sodium phosphate), acetate (e.g., sodium acetate), succinate (e.g., sodium succinate), glutamic acid, glutamate, gluconate, histidine, citrate, or other organic acid buffers. The buffer concentration can be present in a concentration range of about 2 mM to about 1000 mM with a pH in the range of about 4.0 to about 9.0. Examples of pharmaceutically acceptable salts include sodium chloride, sodium acetate and potassium chloride at concentrations of about 2 mM to about 1000 mM.

In one embodiment, the performance-enhancing excipients listed in Table 1, either alone or in the combination with additional excipients, were evaluated for their effect on monomeric species upon thermal stress. In one example, the thermal stress condition was 50° C., the therapeutic protein concentration was about 1 mg/ml to about 500 mg/ml and the performance-enhancing excipient concentration was about 5 mM to about 1000 mM. The formulation was stored at 50° C. for up to 2 weeks. The performance-enhancing excipients from Table 1 were able to reduce the change in monomeric species in comparison to buffer control by at least about 2% and at least about 3% following storage at 50° C. for 1 and 2 weeks, respectively. In another example, the thermal stress condition was 40° C., the therapeutic protein concentration was about 1 mg/ml to about 500 mg/ml and the performance-enhancing excipient concentration was about 5 mM to about 1000 mM. The formulation was stored at 40° C. for up to 4 weeks. The performance-enhancing excipients listed in Table 1 were able to reduce the change in monomeric species in comparison to buffer control by at least 3% and at least about 5% following storage at 40° C. for 2 and 4 weeks, respectively. In another example, the thermal stress condition was 50° C., the therapeutic protein concentration was about 1 mg/ml to about 500 mg/ml and the performance-enhancing excipient concentration was about 5 mM to about 1000 mM. The formulation was stored at 50° C. for up to 2 weeks. The performance-enhancing excipients from Table 1 were able to reduce the change in monomeric species in comparison to buffer control by at least 3% and at least 5% following storage at 50° C. for 1 and 2 weeks, respectively. In another example, the thermal stress condition was 40° C., the therapeutic protein concentration was about 1 mg/ml to about 500 mg/ml and the performance-enhancing excipient concentration was about 5 mM to about 1000 mM. The formulation was stored at 40° C. for up to 8 weeks. The performance-enhancing excipients from Table 1 were able to reduce the change in monomeric species in comparison to buffer control by at least about 2% and at least about 4% following storage at 40° C. for 4 and 8 weeks, respectively.

In another embodiment, the performance-enhancing excipients from Table 1, either alone or in combination with additional excipient(s), were evaluated for their effect on aggregate (HMWS) species upon thermal stress. In one example, the thermal stress condition was 50° C., the therapeutic protein concentration was about 1 mg/ml to about 500 mg/ml and the performance-enhancing excipient concentration was about 5 mM to about 1000 mM. The formulation was stored at 50° C. for up to 2 weeks. The performance-enhancing excipients from Table 1 were able to reduce the change in aggregate content in comparison to change in buffer control by at least about 2% and at least about 3% following storage at 50° C. for 1 and 2 weeks, respectively. In another example, the thermal stress condition was 40° C., the therapeutic protein concentration was about 1 mg/ml to about 500 mg/ml and the performance-enhancing excipient concentration was about 5 mM to about 1000 mM. The formulation was stored at 40° C. for up to 4 weeks. The performance-enhancing excipients from Table 1 were able to reduce the change in aggregate content in comparison to change in buffer control by at least 3% and at least 5% following storage at 40° C. for 2 and 4 weeks. In another example, the thermal stress condition was 50° C., the therapeutic protein concentration was about 1 mg/ml to about 500 mg/ml and the performance-enhancing excipient concentration was about 5 mM to about 1000 mM. The formulation was stored at 50° C. for up to 2 weeks. The performance-enhancing excipients listed in Table 1 were able to reduce the change in aggregate content in comparison to change in buffer control by at least 2% and at least 5% following storage at 50° C. for 1 and 2 weeks, respectively. In another example, the thermal stress condition was 40° C., the therapeutic protein concentration was about 1 mg/ml to about 500 mg/ml and the performance-enhancing excipient concentration was about 5 mM to about 1000 mM. The formulation was stored at 40° C. for up to 8 weeks. The performance-enhancing excipients from Table 1 were able to reduce the change in aggregate content in comparison to change in buffer control by at least about 1% and at least about 4% following storage at 40° C. for 4 and 8 weeks, respectively.

In another embodiment, the performance-enhancing excipients listed in Table 1, either alone or in the combination with additional excipients, were evaluated for their effect on degradant (low molecular weight species, LMWS) species upon thermal stress. In one example, the thermal stress condition was 50° C., the therapeutic protein concentration was about 1 mg/ml to about 500 mg/ml and the performance-enhancing excipient concentration was about 5 mM to about 1000 mM. The formulation was stored at 50° C. for up to 2 weeks. The performance-enhancing excipients from Table 1 were able to reduce the change in degradant content in comparison to change in buffer control by at least about 1% and at least about 2% following storage at 50° C. for 1 and 2 weeks, respectively. In another example, the thermal stress condition was 40° C., the therapeutic protein concentration was about 1 mg/ml to about 500 mg/ml and the performance-enhancing excipient concentration was about 5 mM to about 1000 mM. The formulation was stored at 40° C. for up to 4 weeks. The performance-enhancing excipients from Table 1 were able to reduce the change in degradant content in comparison to change in buffer control by at least about 1% and at least about 2% following storage at 40° C. for 2 and 4 weeks. In another example, the thermal stress condition was 50° C., the therapeutic protein concentration was about 1 mg/ml to about 500 mg/ml and the performance-enhancing excipient concentration was about 5 mM to about 1000 mM. The formulation was stored at 50° C. for up to 2 weeks. The performance-enhancing excipients from Table 1 were able to reduce the change in degradant content in comparison to change in buffer control by at least about 1% and at least about 2% following storage at 50° C. for 1 and 2 weeks. In one example, thermal stress condition was 40° C., the therapeutic protein concentration was about 1 mg/ml to about 500 mg/ml and the performance-enhancing excipient concentration was about 5 mM to about 1000 mM. The formulation was stored at 40° C. for up to 8 weeks. In this example, there was no significant difference in degradant content between formulations containing performance-enhancing excipients from FIG. 1 in comparison to the buffer control following storage at 40° C. for 4 weeks and 8 weeks.

In another embodiment, the performance-enhancing excipients listed in Table 1, either alone or in combination with additional excipients, were evaluated for their effect on charge heterogeneity (acidic peak group, APG) upon thermal stress. In one example, the thermal stress condition was 50° C., the therapeutic protein concentration was about 1 mg/ml to about 500 mg/ml and the performance-enhancing excipient concentration was about 5 mM to about 1000 mM. The formulation was stored at 50° C. for up to 2 weeks. The performance-enhancing excipients from Table 1 were able to reduce the change in APG percent in comparison to change in buffer control by at least about 10% and at least about 20% following storage at 50° C. for 1 and 2 weeks. In another example, the thermal stress condition was 40° C., the therapeutic protein concentration was about 1 mg/ml to about 500 mg/ml and the performance-enhancing excipient concentration was about 5 mM to about 1000 mM. The formulation was stored at 40° C. for up to 4 weeks. The performance-enhancing excipients from Table 1 were able to reduce the change in APG percent in comparison to change in buffer control by at least about 10% and at least about 15% following storage at 40° C. for 2 and 4 weeks. In one example, the thermal stress condition was 50° C., the therapeutic protein concentration was about 1 mg/ml to about 500 mg/ml and the performance-enhancing excipient concentration was about 5 mM to about 1000 mM. The formulation was stored at 50° C. for up to 2 weeks. The performance-enhancing excipients from Table 1 were able to reduce the change in APG percent in comparison to change in buffer control by at least about 5% and at least about 10% following storage at 50° C. for 1 and 2 weeks, respectively. In another example, the thermal stress condition was 40° C., the therapeutic protein concentration was about 1 mg/ml to about 500 mg/ml and the performance-enhancing excipient concentration was about 5 mM to about 1000 mM. The formulation was stored at 40° C. for up to 8 weeks. The performance-enhancing excipients from Table 1 were able to reduce the change in APG percent in comparison to change in buffer control by at least about 10% and at least about 40% following storage at 40° C. for 4 and 8 weeks.

In another embodiment, the performance-enhancing excipients of Table 1, either alone or in combination with additional excipients, were evaluated for their effect on the post translational modification upon thermal stress. In this example, the thermal stress condition was 40° C., the therapeutic protein concentration was about 1 mg/ml to about 500 mg/ml and the performance-enhancing excipient concentration was about 5 mM to about 1000 mM. The formulation was stored at 40° C. for up to 8 weeks. The performance-enhancing excipients were able to prevent the change in asparagine deamidation following 8 weeks of storage at 40° C. During this time, percent deamidation had increased over about 15% in the formulation lacking performance-enhancing excipients.

In another embodiment, the performance-enhancing excipients of Table 1, either alone or in combination with additional excipients, were evaluated for their effect on the protein-protein interaction. In this example, protein-protein interaction was measured as function of protein concentration at 25° C., the therapeutic protein concentration was about 0.001 mg/ml to about 100 mg/ml and the performance-enhancing excipient concentration was about 5 mM to about 1000 mM. The performance-enhancing excipients from Table 1 were able to minimize the protein-protein attractive interaction. Without wanting to be bound to a mechanism of action, it is believed that the protein-protein attractive interaction is responsible for increased viscosity and aggregation with an increase of protein concentration.

A biologic formulation with the performance enhancing excipient (i.e., the enhanced formulation) has superior stability compared to buffer control, has higher monomer retained compared to buffer control upon exposure to stressed temperature conditions, has lower aggregate compared to buffer control upon exposure to stressed temperature conditions, has lowered degradant compared to buffer control upon exposure to stressed temperature conditions, has lower change in percent APG compared to buffer control upon exposure to stressed temperature conditions and/or has a pH between about 4.0 to about 9.0.

### EXAMPLES

### Antibody Production:

The antibody used for the evaluation of the performance-enhancing excipients was manufactured from recombinant CHO-K1, which express a human antibody mAb (IgG1). Cells were grown in CHO medium (Gibco) with 25 μM MSX (Millipore) and 0.1% poloxamer 188 in baffled vented shake flasks. The cultures were incubated at 37° C., at 125 rpm, with 6% CO₂ and >60% humidity. After scaling from 25 ml to 2000 ml over a period of 10-14 days, the culture was used to inoculate 15 liters production vessel (10 liters working volume) containing 7-8 liters CD CHO medium supplemented with l-tyrosine disodium dihydrate (Avantor), Feed C+ (Gibco) and 0.1% poloxamer 188. Cells were inoculated to target an initial cell concentration of 0.7-0.9×10⁶ viable cells/ml. Cultures were grown in fed-batch mode at 37° C. pH was controlled at pH 7±0.2 by sodium carbonate and CO₂. Dissolved oxygen (DO) was controlled at 45±1% in cascade mode by agitation, air and/or O₂ supplementation. Foaming was controlled by the addition of a sterile simethicone antifoam solution as needed. The culture was monitored for viable and total cell concentration using a ViCell analyzer (Beckman). Metabolite utilization, mAb concentration, and waste production were monitored with a Cedex analyzer (Roche). The culture was allowed to grow and produce antibody for a period of 14-17 days in fed batch mode with periodic addition of glucose and 10% Feed C+ (Gibco). At the end of production, the vessel(s) were harvested, centrifuged at 5000×g for 30 minutes. The supernatant was sterile filtered with a 0.2 μM capsule filter into sterile containers and stored at −80° C. until purification by affinity chromatography and final concentration by TFF. Purification was performed using Avantor PROchiev A affinity resin where sample was loaded on a column that was pre-equilibrated in 10 mM sodium phosphate, pH 7.2 buffer (PBS). The mAb was eluted from the column using an elution buffer of 100 mM sodium acetate, pH 3.4. Immediately after elution, the solution was neutralized to pH 7.0 using 2M Tris buffer.

### Synthesis of the Performance-Enhancing Excipients:

The amino acids are first treated with sodium bicarbonate and then reacted with the appropriate alkanoic acid anhydride in an aqueous solution. The reaction mixture is worked up by adjusting the aqueous solution to pH 2 and then extracting with ethyl acetate. The product is then purified to give crystals of acceptable purity.

### Sample Preparation for Viscosity Measurement:

mAb stock at ˜200 mg/ml was buffer exchanged into the desired formulations using an Amicon Ultracel 50K centrifugal filter device. The material was buffer exchanged with 5× volume of desired buffer system and then further concentrated using a Beckman Coulter centrifuge at 3800×g. The protein concentration of the concentrated material was then determined. For viscosity measurement, buffer exchanged material was concentrated to 300 mg/ml and 250 mg/ml. The formulation conditions for viscosity measurement at 300 mg/ml and 250 mg/ml are shown in Table 2 and Table 3, respectively.

TABLE 2
Formulations at 300 mg/ml mAb concentration
for viscosity measurements

| Formulation | Composition |
|---|---|
| Buffer | 300 mg/ml mAb, 10 mM sodium phosphate pH 8.0 |
| Sodium | 300 mg/ml mAb, 10 mM sodium phosphate pH 8.0, |
| Chloride | 300 mM sodium chloride |
| Histidine | 300 mg/ml mAb, 10 mM sodium phosphate pH 8.0, |
| | 300 mM Histidine |
| Acetyl | 300 mg/ml mAb, 10 mM sodium phosphate pH 8.0, |
| Histidine | 300 mM Acetyl Histidine |
| Propionyl | 300 mg/ml mAb, 10 mM sodium phosphate pH 8.0, |
| Histidine | 300 mM Propionyl Histidine |
| Arginine | 300 mg/ml mAb, 10 mM sodium phosphate pH 8.0, |
| | 300 mM Arginine |
| Acetyl | 300 mg/ml mAb, 10 mM sodium phosphate pH 8.0, |
| Arginine | 300 mM Acetyl Arginine |
| Propionyl | 300 mg/ml mAb, 10 mM sodium phosphate pH 8.0, |
| Arginine | 300 mM Propionyl Arginine |
| Serine | 300 mg/ml mAb, 10 mM sodium phosphate pH 8.0, |
| | 300 mM Serine |
| Acetyl | 300 mg/ml mAb, 10 mM sodium phosphate pH 8.0, |
| Serine | 300 mM Acetyl Serine |
| Propionyl | 300 mg/ml mAb, 10 mM sodium phosphate pH 8.0, |
| Serine | 300 mM Propionyl Serine |
| Lysine | 300 mg/ml mAb, 10 mM sodium phosphate pH 8.0, |
| | 300 mM Lysine |
| Acetyl | 300 mg/ml mAb, 10 mM sodium phosphate pH 8.0, |
| Lysine | 300 mM Acetyl Lysine |
| Propionyl | 300 mg/ml mAb, 10 mM sodium phosphate pH 8.0, |
| Lysine | 300 mM Propionyl Lysine |
| Bis Acetyl | 300 mg/ml mAb, 10 mM sodium phosphate pH 8.0, |
| Lysine | 300 mM Bis Acetyl Lysine |
| Bis Propionyl | 300 mg/ml mAb, 10 mM sodium phosphate pH 8.0, |
| Lysine | 300 mM Bis Propionyl Lysine |

TABLE 3
Formulations at 250 mg/ml mAb concentration
for viscosity measurements

| Formulation | Composition |
|---|---|
| Buffer | 250 mg/ml mAb, 10 mM sodium phosphate pH 8.0 |
| Sodium | 250 mg/ml mAb, 10 mM sodium phosphate pH 8.0, |
| chloride | 300 mM sodium chloride |
| Arginine | 250 mg/ml mAb, 10 mM sodium phosphate pH 8.0, |
| | 300 mM Arginine |
| Acetyl | 250 mg/ml mAb, 10 mM sodium phosphate pH 8.0, |
| Arginine | 300 mM Acetyl Arginine |
| Propionyl | 250 mg/ml mAb, 10 mM sodium phosphate pH 8.0, |
| Serine | 300 mM Propionyl Serine |
| Bis Acetyl | 250 mg/ml mAb, 10 mM sodium phosphate pH 8.0, |
| Lysine | 300 mM Bis Acetyl Lysine |
| Bis Acetyl | 250 mg/ml mAb, 10 mM sodium phosphate pH 8.0, |
| Lysine:Propio- | 150 mM Bis Acetyl Lysine, 150 mM Propionyl Serine |
| nyl Serine (1:1) | |

### Sample Preparation for Stability Measurement:

mAb stock at ˜200 mg/ml was buffer exchanged into the desired formulations using an Amicon Ultracel 50K centrifugal filter device. The material was buffer exchanged with 5× volume of desired buffer system and then further concentrated using a Beckman Coulter centrifuge at 3800×g. The protein concentration on the concentrated material was then determined. Buffer exchanged material was concentrated to 250 mg/ml or diluted to 10 mg/ml with a matching buffer. The formulation conditions for the stability study at 250 mg/ml and 10 mg/ml are shown in Tables 4 and Table 5. The buffer exchanged material was then aliquoted into 2 ml glass vials where each vial contained approximately 0.7 ml of sample. The sample aliquots were then placed on a stability station according to Table 6 and analyzed by SEC-HPLC and IEC-HPLC at the predetermined time points shown in Table 6.

TABLE 4
Formulations at 250 mg/ml mAb concentration for stability study

| Formulation | Composition |
|---|---|
| Buffer | 250 mg/ml mAb, 10 mM sodium phosphate pH 8.0 |
| Arginine | 250 mg/ml mAb, 10 mM sodium phosphate pH 8.0, |
| | 300 mM Arginine |
| Propionyl | 250 mg/ml mAb, 10 mM sodium phosphate pH 8.0, |
| Serine (PS) | 300 mM Propionyl Serine |
| Bis Acetyl | 250 mg/ml mAb, 10 mM sodium phosphate pH 8.0, |
| Lysine (BAL) | 300 mM Bis Acetyl Lysine (BAL) |
| PS:BAL (1:1) | 250 mg/ml mAb, 10 mM sodium phosphate pH 8.0, |
| | 150 mM BisAcetyl Lysine, 150 mM Propionyl Serine |

TABLE 5
Formulations at 10 mg/ml mAb concentration for stability study

| Formulation | Composition |
|---|---|
| Buffer | 10 mg/ml mAb, 10 mM sodium phosphate pH 8.0 |
| Sodium | 10 mg/ml mAb, 10 mM sodium phosphate pH 8.0, |
| Chloride | 300 mM sodium chloride |
| Mannitol | 10 mg/ml mAb, 10 mM sodium phosphate pH 8.0, |
| | 300 mM Mannitol |
| Sucrose | 10 mg/ml mAb, 10 mM sodium phosphate pH 8.0, |
| | 300 mM Sucrose |
| Glycine | 10 mg/ml mAb, 10 mM sodium phosphate pH 8.0, |
| | 300 mM Glycine |
| Arginine | 10 mg/ml mAb, 10 mM sodium phosphate pH 8.0, |
| | 300 mM Arginine |
| Propionyl | 10 mg/ml mAb, 10 mM sodium phosphate pH 8.0, |
| serine (PS) | 300 mM Propionyl Serine (PS) |
| Bis Acetyl | 10 mg/ml mAb, 10 mM sodium phosphate pH 8.0, |
| Lysine (BAL) | 300 mM Bis Acetyl lysine (BAL) |
| PS:BAL (1:1) | 10 mg/ml mAb, 10 mM sodium phosphate pH 8.0, |
| | 150 mM BAL + 150 mM PS |

TABLE 6
Stability conditions and time points

| mAb | | Time points (weeks) | | |
|---|---|---|---|---|
| concentration | | 4° C. stability | 40° C. stability | 50° C. stability |
| (mg/ml) | | station | station | station |
| 250 | mg/ml | 0, 1, 2, 4 | 2, 4 | 1, 2 |
| 10 | mg/ml | 0, 1, 2, 4, 8 | 4, 8 | 1, 2 |

### Sample Preparation for LC-MS/MS Measurement:

mAb stock at ˜200 mg/ml was buffer exchanged into the desired formulations using an Amicon Ultracel 50K centrifugal filter device. The material was buffer exchanged with 5× volume of desired buffer system and then further concentrated using a Beckman Coulter centrifuge at 3800×g. The protein concentration on the concentrated material was then determined. Buffer exchanged material was diluted to 10 mg/ml with a matching buffer. The formulation conditions for the LC-MS/MS analysis are shown in Table 7. The buffer exchanged material was then aliquoted into 2 ml glass vials where each vial contained approximately 0.7 ml of sample. The sample aliquots were then placed on a 40° C. stability station for 8 weeks. Following the intended storage period, control, and 40° C. samples were analyzed by LC-MS/MS for post translational modifications.

TABLE 7
Formulations and storage conditions for LC-MS/MS analysis

| Formulation | Composition/storage condition |
|---|---|
| Buffer, | 10 mg/ml mAb, 10 mM sodium phosphate pH |
| 4° C.-8 weeks | 8.0/4° C.-8 weeks storage |
| Buffer, | 10 mg/ml mAb, 10 mM sodium phosphate pH |
| 40° C.-8 weeks | 8.0/40° C.-8 weeks storage |
| Bis Acetyl Lysine, | 10 mg/ml mAb, 10 mM sodium phosphate pH 8.0, |
| 40° C.-8 weeks | 300 mM Bis Acetyl Lysine, 40° C.-8 weeks storage |
| Propionyl Serine, | 10 mg/ml mAb, 10 mM sodium phosphate pH 8.0, |
| 40° C.-8 weeks | 300 mM Propionyl Serine, 40° C.-8 weeks storage |

### Sample Preparation for DLS Measurement:

The buffers were filtered through 0.22 μm sterile filters and used to dilute the filtered antibody solution stocks (10 mg/ml) to concentrations ranging between 1.0 mg/ml and 12.5 mg/ml. All dilutions were prepared in duplicate.

### Viscosity Determination:

Viscosity was measured at 300 mg/ml and 250 mg/ml mAb concentrations. A circulating water bath for the Brookfield DVII+ viscometer was set to 25° C. and warmed for approximately 1 hour prior to sample testing. First, the viscosity of the standard solutions B29 (Brookfield, viscosity 29 cp), RT100 (Cannon Instrument, viscosity 96 cp) and RT500 (Cannon Instrument, viscosity 480 cp) was measured to confirm that the instrument was calibrated for the viscosity range of samples. Sample measurements were taken similarly at a volume of 0.6 ml at 25° C. For each condition, 2 measurements were obtained and the average viscosity along with standard deviation was reported.

### Size-Exclusion Chromatography Analysis (SEC-HPLC):

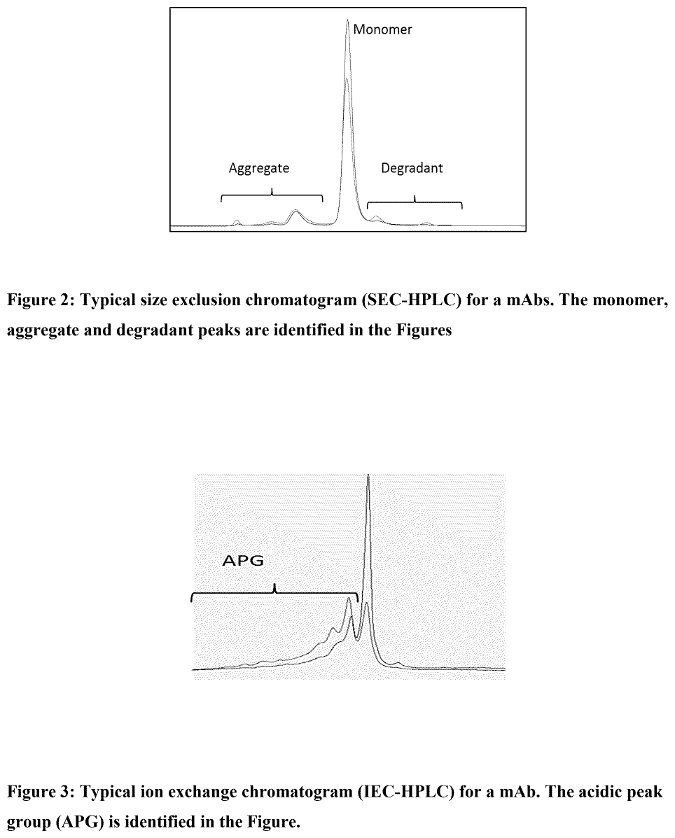

SEC-HPLC analysis was performed to determine the change in size variance (% monomer, aggregate and degradant) as a result of thermal stress. Samples were taken out from the stability stations at the predetermined time points as shown in Table 6, diluted with phosphate buffer saline to 5 mg/ml and then loaded onto a Tosoh Bioscience HPLC Column. The sample loading and elution buffer was 10 mM sodium phosphate, 500 mM cesium chloride, pH 7.0. The flow rate was 0.3 ml/min, and the column temperature was maintained to 25° C. A typical SEC-HPLC chromatogram is shown in FIG. 2. The percents monomer, aggregate and degradant for test samples were calculated as shown in FIG. 2.

### Ion Exchange Chromatography Analysis (IEC-HPLC):

IEC-HPLC analysis was performed to determine the change in charge variance (% acidic peak group, APG) as a result of thermal stress. Monoclonal antibodies are heterogeneous in nature, and acid peak groups (APG) are variants of the antibody that have lower apparent isoelectric points (pl) than the primary variant. APG elute prior to the main peak on IEC-HPLC. Samples were taken out from the stability stations at the predetermined time points, diluted with phosphate buffer saline to 5 mg/ml and then loaded onto an HPLC Thermo ProPac WCX-10 column that was pre-equilibrated with 10 mM sodium phosphate, pH 7.8 buffer. The sample was eluted with a salt gradient (0-200 mM sodium chloride) in a 10 mM sodium phosphate, pH 7.8 buffer. The flow rate was 1.0 ml/min, and the column temperature was maintained at 30° C. A typical IEC-HPLC chromatogram is shown in FIG. 3. The percent APG for test samples was calculated as shown in FIG. 3.

### Lc-Ms/Ms Analysis:

The free thio groups of proteins were blocked by adding N-ethylmaleimide (NEM). The excess NEM reagent was removed by protein precipitation using ethanol/chloroform method. The protein pellet was resuspended in lysis buffer containing 8 M urea and 50 mM Tris (pH 7.5). The protein was reduced by DTT and alkylated by IAM prior to in-solution trypsin digestion. The resultant peptides were C18 desalted and direct analyzed by LC-MS/MS on Orbitrap Fusion Lumos MS instrument using OTOT method. The MS/MS spectra were searched against UniProt human database plus the protein sequence HC and LC using Sequest search engines on Proteome Discoverer (V2.4) platform and post translational modifications were analyzed.

### Dynamic Light Scattering (DLS)

A Zetasizer Nano ZS Series instrument (Malvern Panalytical Ltd., Malvern, UK) was used to measure dynamic light scattering (DLS) of the molecules. DLS measurements were performed at 25° C. in triplicate for each sample, with automatic detection of number of runs. Data generated was given an average size distribution by number of molecules in the sample. A 633 nm He—Ne laser was utilized, and light scattering analyzed by an avalanche photodiode. Backscatter signal at 173° was picked to minimize contribution from dust. DTS (Version 4.2) software (Malvern Panalytical Ltd., Malvern, UK) was used to acquire and deconvolute the autocorrelogram. The diffusion coefficient of the major peak at a diameter of about 10 nm from the DLS measurements corresponding to the mAb monomer was applied to obtain the mutual diffusion coefficient (Dₘ). At relatively low protein concentrations, Dₘ can be related to the sample concentration (c) (g/mL), the interaction parameter (k_{D}) (ml/g), and the self-diffusion coefficient (Dₛ) (diffusion of a single molecule, in the limit of infinite dilution, measured in μm²/sec), as follows: Dₘ=Dₛ(1+k_{D}c)

k_{D} can be obtained from the ratio of slope to intercept in a Dₘ versus c plot. The value of k_{D} can be used to describe the nature of intermolecular interactions, with a positive k_{D} signifying intermolecular repulsions, and negative k_{D} representing attractive interactions. For each preparation, the protein concentration was verified, and the measured concentration values were used to calculate k_{D}.

### Example 1: Solution Viscosity Reduction by Performance-Enhancing Excipients

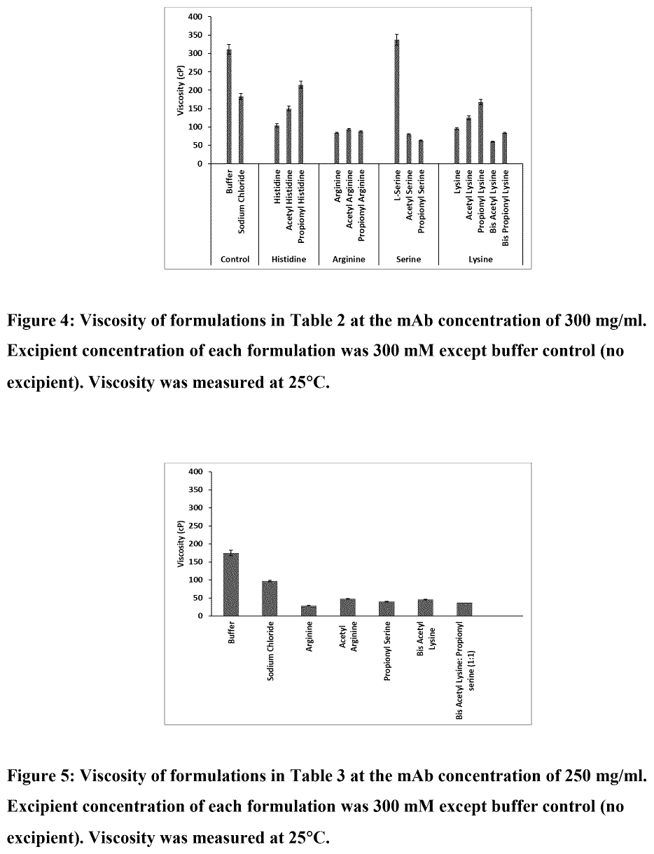

The effect of amino acids (histidine, arginine, serine, and lysine) and their derivatives, performance-enhancing excipients (acetyl, propionyl, bis acetyl, bis propionyl), on the viscosity of mAb was evaluated at the 300 mg/ml and 250 mg/ml monoclonal antibody (mAb) concentrations in a 10 mM phosphate buffer at pH 8.0. The goal of the study was to evaluate if the performance-enhancing excipients are able to reduce the viscosity of mAb solution. Viscosity reduction by amino acids and their derivatives (performance-enhancing excipients) was also compared with a buffer control (10 mM sodium phosphate pH 8.0) and 10 mM sodium phosphate buffer containing 300 mm sodium chloride at pH 8.0. As shown in FIG. 4, each derivative has a different effect on viscosity reduction. In the case of histidine and lysine, the acetyl and propionyl derivatives resulted in a decrease in the ability to reduce the solution viscosity of the mAb solution. In the case of arginine, derivatization has no significant impact. However, in the case of serine, both acetyl and propionyl derivatives have significantly enhanced the viscosity reducing ability of serine. However, propionyl serine performed better than acetyl serine. Acetyl and propionyl derivatives have reduced the ability of lysine to reduce viscosity; however, bis acetyl and bis propionyl derivatives have demonstrated better viscosity reduction compared to lysine. Among all tested formulations, propionyl serine and bis acetyl lysine performed best in terms of viscosity reduction at 300 mM concentration. Both were able to reduce viscosity by about 80% compared to the control (10 mM sodium phosphate at 8.0).

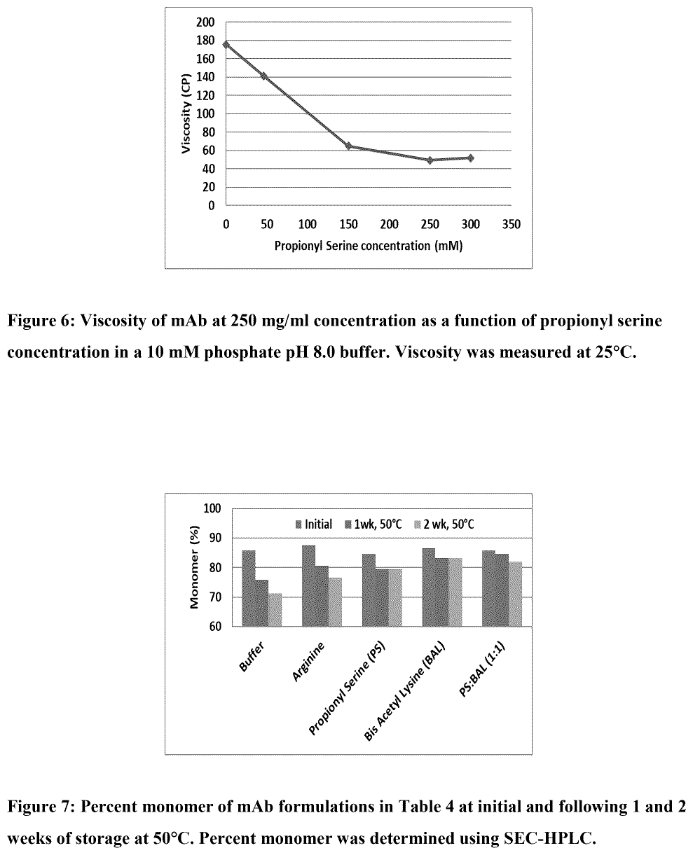

The viscosity reduction of selected performance-enhancing excipients was also tested at 250 mg/ml. The effect of selected excipients on the viscosity of mAbs at 250 mg/ml in 10 mM sodium phosphate buffer at pH 8.0 is shown in FIG. 5. The performance of propionyl serine and bis acetyl lysine was comparable to those seen as 300 mg/ml. Both were able to reduce the viscosity by about 75%. Interestingly, the performance of 50%:50% mixture of propionyl serine and bis acetyl lysine was better than propionyl serine or bis acetyl lysine alone, demonstrating a synergistic effect. The 50%:50% (150 mM each) mixture of propionyl serine and bis acetyl lysine has reduced viscosity by approximately 80% compared to 75% by either propionyl serine or bis acetyl lysine (300 mM). The viscosity of 250 mg/ml mAb was also measured as a function of propionyl serine. Results in FIG. 6 demonstrate that viscosity reduction is dependent on excipient concentration.

### Example 2: Stability of Therapeutic Protein at 250 mg/Ml at 50° C.

The effect of selected formulations in Table 4 on the thermal stability of mAbs was measured at 250 mg/ml mAb concentration. Sample preparation was performed by buffer exchanging a mAb stock, originally in Tris buffer at pH 7.5, into the buffers listed in Table 4. mAb stock material was buffer exchanged with 5× volume of each desired buffer system and then further concentrated using a Beckman Coulter centrifuge at 3800×g. The protein concentration on the concentrated material was then determined and adjusted to 250 mg/ml. Buffer exchanged formulations were then aliquoted into 2 ml glass vials. Each vial contained approximately 0.7 ml of sample. Formulation samples were then placed at a 50° C. stability station for 2 weeks. The initial and stability samples were analyzed by Size-Exclusion and Ion-Exchange Chromatography at the predetermined time points shown in Table 6.

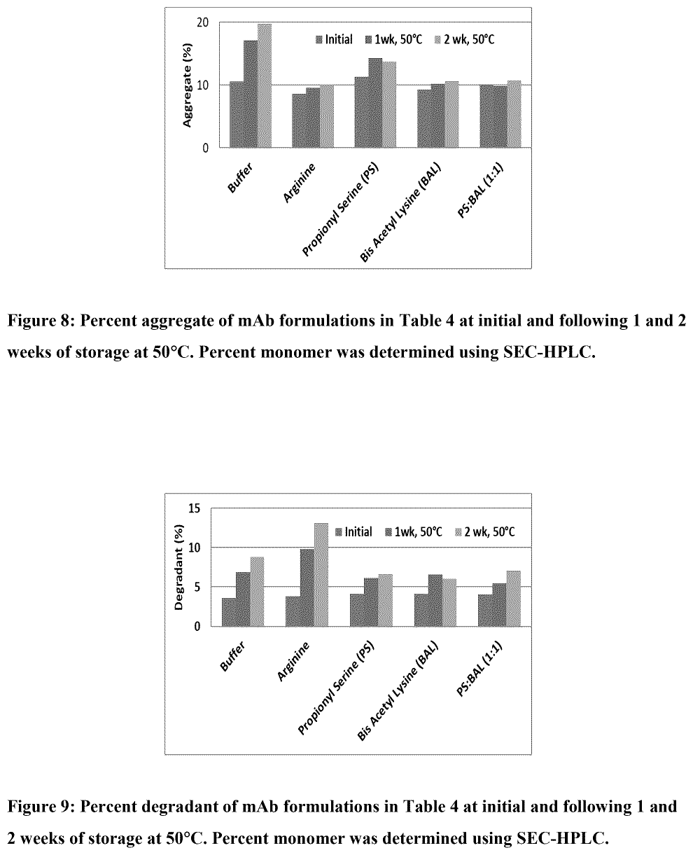

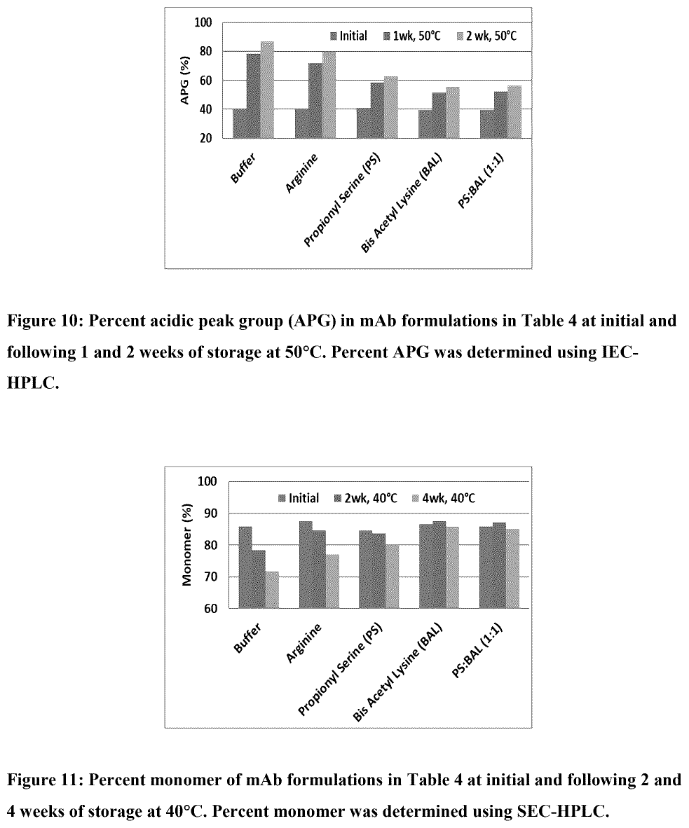

The percent monomer, aggregate, degradant and APG for initial and 1 week and 2 weeks samples following storage at 50° C. are shown in FIG. 7, FIG. 8, FIG. 9, and FIG. 10, respectively. All formulations containing performance-enhancing excipients listed in Table 1 were superior compared to the buffer control; however, the best formulations were bis acetyl lysine containing formulations, followed by the formulation containing propionyl serine.

Percent monomer remaining following 2 weeks of storage at 50° C. was about 12% and 8% higher in bis acetyl lysine and propionyl serine compared to the buffer control, respectively. Percent monomer remaining for bis acetyl lysine and propionyl serine containing formulations was better in comparison to arginine by 6.5% and 3%, respectively.

Percent aggregate in formulations containing bis acetyl lysine and propionyl serine following 2 weeks of storage at 50° C. was about 9% and 6% lower than in the buffer control, respectively. Percent aggregate in bis acetyl lysine and arginine containing formulations was comparable and slightly lower than the formulation containing propionyl serine.

Percent degradant in the formulations containing bis acetyl lysine and propionyl serine following 2 weeks of storage at 50° C. was about 2% lower compared to percent degradant in the buffer control. Interestingly, arginine increased the degradant content in the control formulation by 4.5%. The formulations containing bis acetyl lysine and propionyl serine have about 6% lower degradant compared to the formulation containing arginine following 2 weeks of storage at 50° C.

The biggest difference among formulations was seen in the acid peak group (APG) content following 2 weeks of storage at 50° C. The percent APG increased in all the formulations after storage; however, the change was significantly lower in formulations containing bis acetyl lysine and propionyl serine compared to buffer control and arginine containing formulations. The change in percent APG following 2 weeks of storage at 50° C. was about 31% and 24% lower in bis acetyl lysine and propionyl serine formulations compared to the buffer control, respectively. The percents APG for bis acetyl lysine and propionyl serine formulations were also better compared to arginine by 24% and 16%, respectively.

### Example 3: Stability of Therapeutic Protein at 250 mg/Ml at 40° C.

The effect of selected formulations in Table 4 on thermal stability was measured at 40° C. The mAb concentration was 250 mg/ml in all formulations. Sample preparation was performed by buffer exchanging a mAb stock (originally in Tris buffer at pH 7.5) into the buffers listed in Table 4. Stock material was buffer exchanged with 5× volume of desired buffer system and then further concentrated using a Beckman Coulter centrifuge at 3800×g. The protein concentration on the concentrated material was then determined and adjusted to 250 mg/ml. Buffer exchanged formulations were then aliquoted into 2 ml glass vials. Each vial contained approximately 0.7 ml of sample. Formulation samples were then placed on a 40° C. stability station for 2 and 4 weeks. The initial and stability samples were analyzed by Size-Exclusion and Ion-Exchange Chromatography at predetermined time points as shown in Table 6.

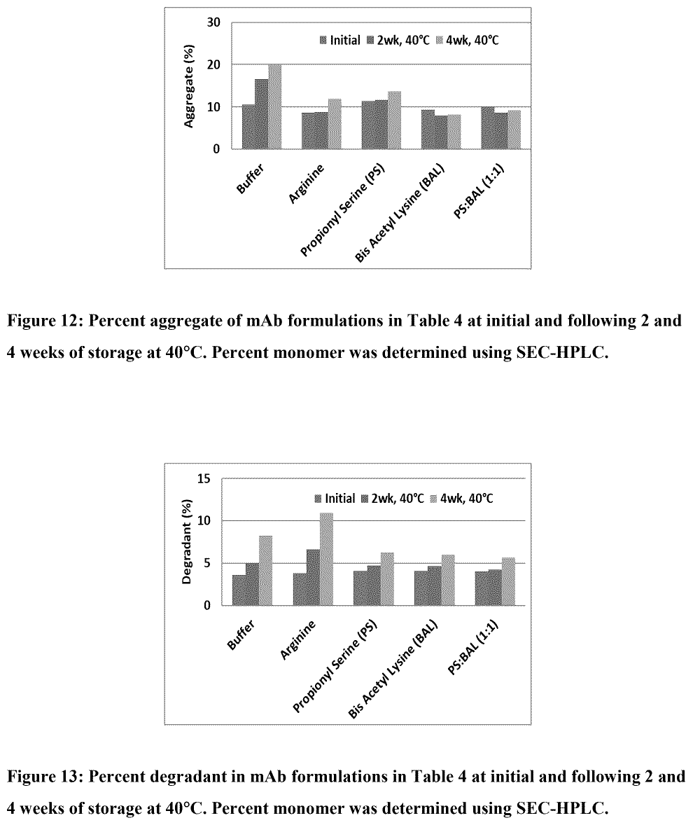

The percents monomer, aggregate, degradant and APG for initial and following 2 week and 4 weeks of storage at 40° C. are shown in FIG. 11, FIG. 12, FIG. 13, and FIG. 14, respectively. All formulations containing performance-enhancing excipients listed in Table 1 were superior compared to the buffer control; however, the best formulations were those containing bis acetyl lysine, followed by propionyl serine.

Percent monomer remaining following 4 weeks of storage at 40° C. was about 14% and 8% higher in formulations containing bis acetyl lysine and propionyl serine compared to the buffer control and was about 9% and 3% higher compared to arginine, respectively.

Percent aggregate following 4 weeks of storage at 40° C. was about 12% and 6% lower in formulations containing bis acetyl lysine and propionyl serine compared to the buffer control, respectively. Percent aggregates in the bis acetyl lysine formulation was about 4% lower compared to the arginine formulation.

Percent degradant in the formulations containing bis acetyl lysine and propionyl serine following 4 weeks of storage at 40° C. was about 2% lower compared to degradant in the buffer control. Interestingly, arginine has increased the degradant content in the control formulation by 3%. The formulations containing bis acetyl lysine and propionyl serine have about 5% lower degradant compared to the formulation containing arginine.

The biggest difference seen amongst the formulations was in regard to the percent acidic peak group (APG). The percent APG increased in all the formulations following storage at 40° C.; however, the change was significantly lower in formulations containing bis acetyl lysine and propionyl serine compared to the buffer control. The change in percent APG following 4 weeks of storage at 40° C. was about 38% and 26% lower in formulations containing bis acetyl lysine and propionyl serine compared to the buffer control, respectively and about 26% and 13% lower compared to arginine formulations, respectively.

### Example 4: Stability of Therapeutic Protein at 10 mg/Ml at 50° C.

The effect of formulation excipients in Table 5 on the thermal stability of mAbs was measured at 10 mg/ml mAb concentration. Sample preparation was performed by buffer exchanging a mAb stock in Tris buffer at pH 7.5 into the buffers listed in Table 5. The stock material was buffer exchanged with 5× volume of desired buffer system and then diluted with matching buffer to obtain a final concentration to 10 mg/ml. Buffer exchanged formulations were then aliquoted into 2 ml glass vials. Each vial contained approximately 0.7 ml of sample. Formulation samples were then placed on a 50° C. stability station for 1 and 2 weeks. The initial and stability samples were analyzed by Size-Exclusion and Ion-Exchange Chromatography at predetermined time points as shown in Table 6.

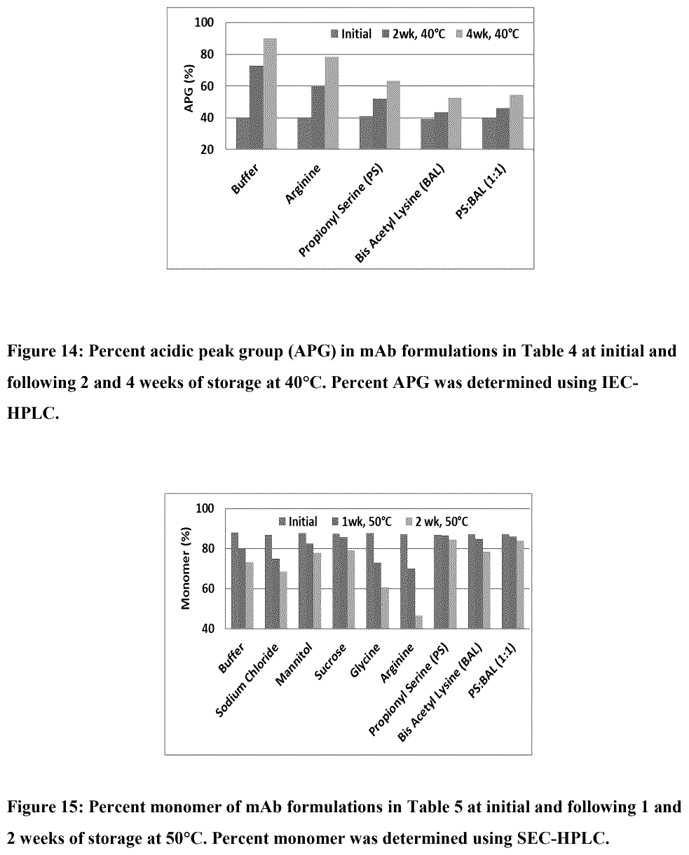

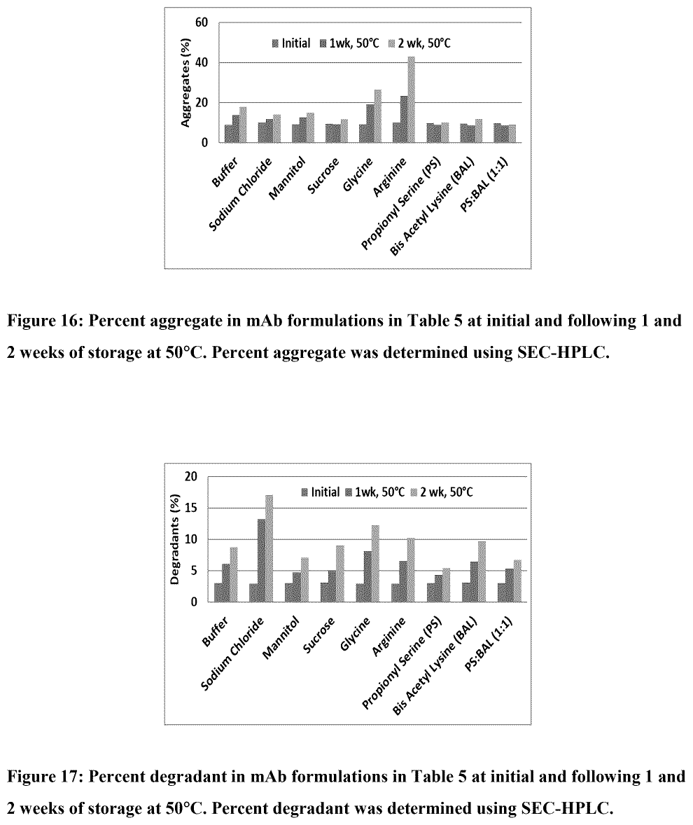

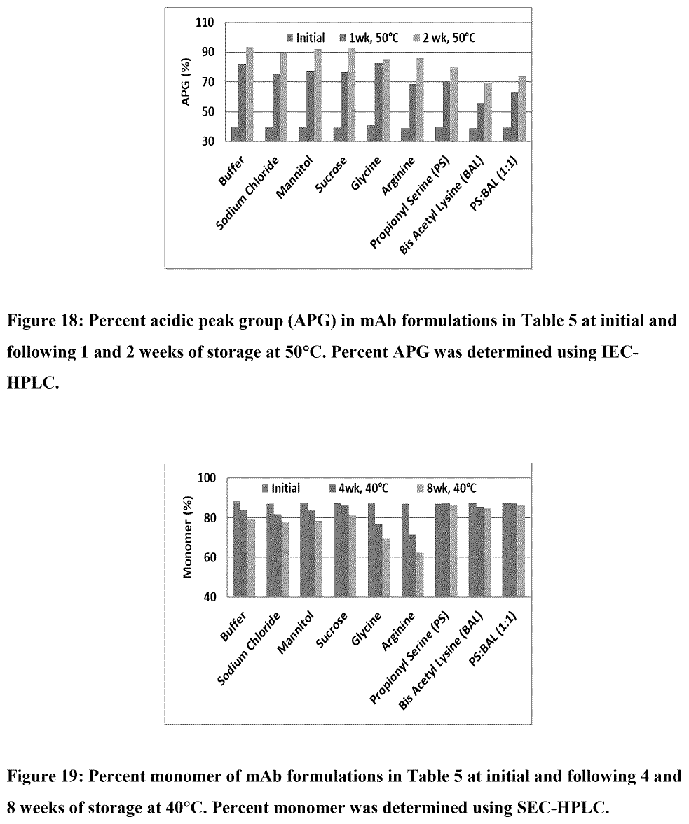

The percents monomer, aggregate, degradant and APG for initial and following 1 week and 2 weeks of storage at 50° C. are shown in FIG. 15, FIG. 16, FIG. 17, and FIG. 18, respectively. The size variance (monomer, aggregate, degradant) was measured by SEC-HPLC, and charge variance (APG) was measured by IEC-HPLC. Percent monomer decrease and percent aggregate and degradant increase occurred in all formulation over time. The % APG also increased in all formulations with time.

Percent monomer remaining in formulations following 2 weeks of storage at 50° C. was largest in formulations containing bis acetyl lysine followed by propionyl serine and sucrose. Percent monomer remaining in bis acetyl lysine and propionyl serine formulations was about 11% and 5% higher compared to the buffer control, respectively. Except for sucrose, no other formulation was even close to bis acetyl lysine and propionyl serine in terms of percent monomer retained. Arginine was the worst performer amongst all tested formulations following 2 weeks of storage at 50° C.

Percent aggregate following 2 weeks of storage at 50° C. was lowest in the formulation containing bis acetyl lysine, followed by propionyl serine and sucrose in comparison to all other formulations. Percent aggregate in bis acetyl lysine, propionyl serine and sucrose formulations was at least 6% lower compared to the buffer control. Arginine was the worst performer amongst all tested formulations following 2 weeks of storage at 50° C.

Percent degradant was not a leading differentiator among formulations, although propionyl serine performed better than all other formulations tested following 2 weeks of storage at 50° C.

The biggest difference amongst formulations was seen in regard to the percent acid peak group (APG). Percent APG increased in all formulations following storage at 50° C.; however, the change in percent increase was significantly lower in formulations containing bis acetyl lysine and propionyl serine in comparison to other formulations. Percent APG following 2 weeks of storage at 50° C. was about 20% and 14% lower in bis acetyl lysine and propionyl serine containing formulations compared to the buffer control, respectively. Sucrose, which performed well in protecting against change in size variance, did not protect against change in charge variance.

### Example 5: Stability of Therapeutic Protein at 10 mg/Ml at 40° C.

The effect of formulation excipients in Table 5 on the thermal stability of mAbs was measured at 10 mg/ml mAb concentration. Sample preparation was performed by buffer exchanging mAb stock in Tris buffer at pH 7.5 into the buffers listed in Table 5. The stock material was buffer exchanged with 5× volume of desired buffer system and then diluted with matching buffer to obtain a final concentration of 10 mg/ml. Buffer exchanged formulations were then aliquoted into 2 mL glass vials. Each vial contained approximately 0.7 ml of sample. Formulation samples were then placed on a 40° C. stability station for 4 and 8 weeks. The initial and stability samples were analyzed by Size-Exclusion and Ion-Exchange Chromatography at predetermined time points as shown in Table 6. The selected samples (Table 7) were also analyzed by LC-MS/MS.

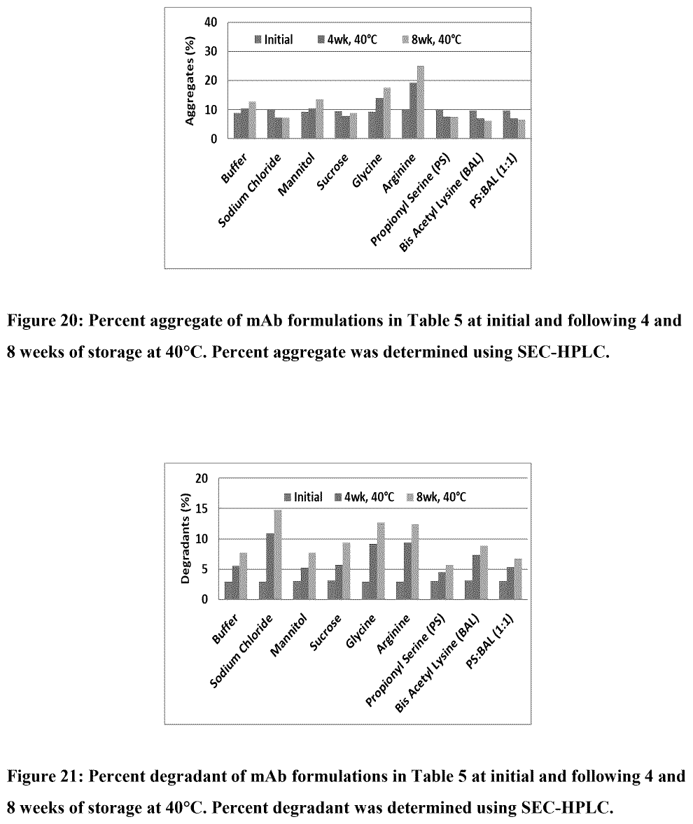

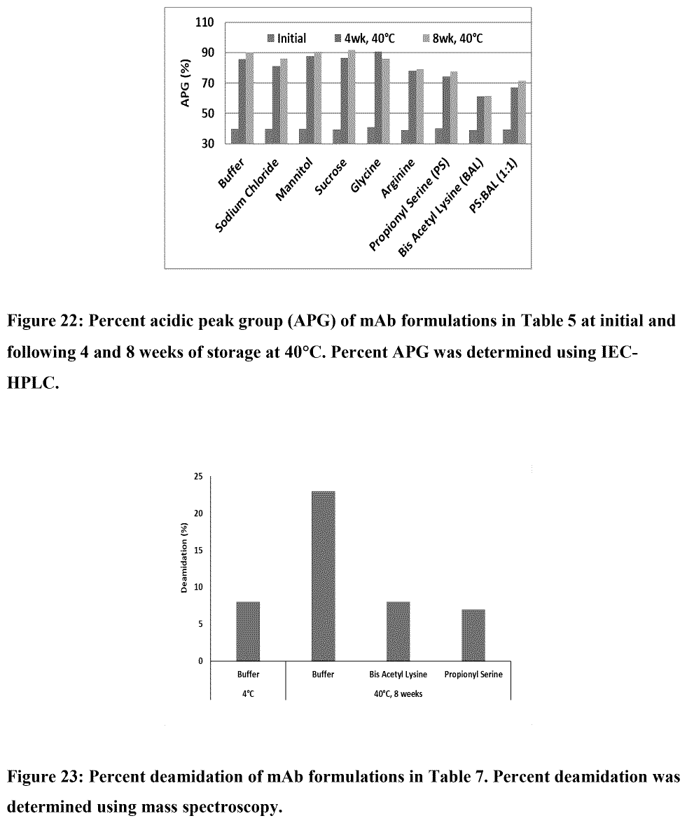

The percent monomer, aggregate, degradant and APG for initial and following 4 weeks and 8 weeks of storage at 40° C. are shown in FIG. 19, FIG. 20, FIG. 21, and FIG. 22, respectively. The % monomer decrease and % aggregate and degradant increase occurred in all formulations over time. The % APG also increased in all formulations with time.

Percent monomer remaining in the formulations following 8 weeks of storage at 40° C. was largest in formulations containing bis acetyl lysine and propionyl serine followed by sucrose. Percent monomer remaining in bis acetyl lysine and propionyl serine formulations was about 5% and 7% higher compared to the buffer control, respectively. Except for sucrose, no other formulation was even close to bis acetyl lysine and propionyl serine in terms of percent monomer remaining following 8 weeks of storage. Arginine was the worst performer amongst all the tested formulations. Percent monomer remaining in the bis acetyl lysine and propionyl serine formulations was about 22% and 24% higher compared to arginine containing formulations, respectively.

Percent aggregate following 8 weeks of storage at 40° C. was lower in bis acetyl lysine, followed by propionyl serine, NaCl, and sucrose compared to all other formulations. Percent aggregate in bis acetyl lysine and propionyl serine formulations was at least 5% lower than the control buffer formulation. Arginine and glycine were the worst performers amongst all formulations, where the increase in percent aggregate following 8 weeks of storage at 40° C. was about 12% and 5% higher than the control, respectively, and about 19% and 11% higher than in bis acetyl lysine and propionyl serine formulations, respectively.

Percent degradant was not a leading differentiator amongst formulations, although propionyl serine performed better than all other formulations tested following 8 weeks of storage at 40° C. Sodium chloride, arginine and glycine were amongst the worst performers, where the increase in percent degradant was about 4% higher than the control, bis acetyl lysine and propionyl serine containing formulations.

The biggest difference among formulations was in the percent acid peak group (APG) content. The percent APG increased in all the formulations following storage at 40° C.; however, the change was significantly lower in formulations containing bis acetyl lysine and propionyl serine in comparison to the other formulations. Percent APG following 8 weeks of storage at 40° C. was about 29% and 13% lower in formulations containing bis acetyl lysine and propionyl serine compared to the buffer control, respectively. Sucrose, which performed well in protecting against change in size variance, did not protect against a change in charge variance.

The results of LC-MS/MS analysis of samples in Table 7 are shown in FIG. 23. LS-MS/MS analysis demonstrated two deamidation sites in the heavy chain of the antibody. The buffer control sample that was stored at 4° C. had 8% deamination. Amongst the samples stored at 40° C. for 8 weeks, percent deamination was 23%, 8% and 7% in the buffer control, bis acetyl lysine and propionyl serine formulations, respectively. These results clearly demonstrate that the bis acetyl lysine and propionyl serine can control the asparagine deamidation of the antibody. The potential mechanism by which bis acetyl lysine and propyl serine are able to protect the antibody against asparagine deamidation could be via H-binding of the side chain of asparagine with bis acetyl lysine and propionyl serine. This observation compliments the reduction in % APG change in bis acetyl lysine and propionyl serine formulations stored at 40° C. in example 5. During the deamidation process, asparagine is converted into aspartic acid, which is more acidic than asparagine.

### Protein-Protein Interaction Measurement Using DLS

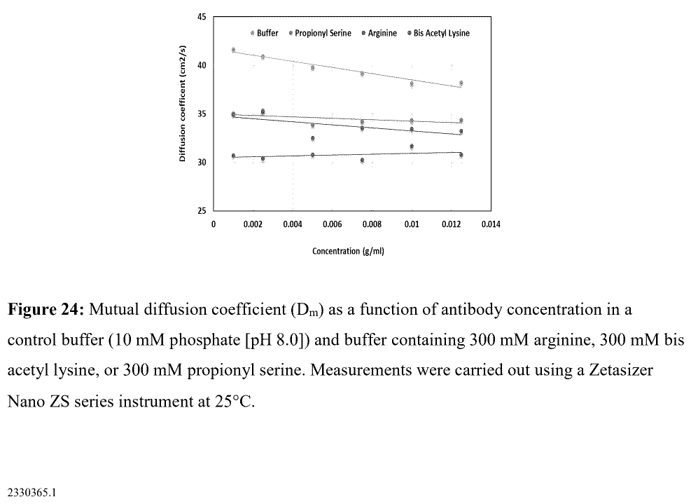

Protein-protein interactions were measured in buffer alone (10 mM phosphate [pH 8.0]) and buffer containing arginine, bis acetyl lysine, and propionyl serine. The Dₘ was measured as a function of antibody concentration, ranging from 1 mg/ml (0.001 g/ml) to 12.5 mg/ml (0.0125 g/ml). As shown in FIG. 24 and Table 8, negative Dₘ slopes for buffer, arginine, and propionyl serine suggest the presence of protein-protein attractive interactions in these formulations. Attractive interaction was weakest in propionyl serine, followed by arginine, and buffer. The formulation containing bis acetyl lysine demonstrated protein-protein repulsive interaction.

TABLE 8
Interaction parameter (k_{D}) by formulation

| Formulation | k_{D} (ml/g) |
|---|---|
| Buffer (10 mM phosphate pH 8.0) | −8.53 |
| Buffer w/300 mM Arginine | −4.54 |
| Buffer w/300 mM Propionyl Serine | −3.11 |
| Buffer w/300 mM Bis Acetyl Lysine | 2.39 |

1. A biologic formulation comprising a performance-enhancing excipient comprising a compound having the following chemical structure: 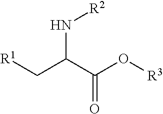
wherein,
R¹═OH—, OCOCH₃, OCOC₂H₅, OCOC₃H₇, OCOC₄H₉, OCOC₅H₁₁, OCOC₆H₁₃, CH₃CONHCH₂CH₂CH₂, C₂H₅CONHCH₂CH₂CH₂, C₃H₇CONHCH₂CH₂CH₂, C₄H₉CONHCH₂CH₂CH₂, C₅H₁₁CONHCH₂CH₂CH₂, C₆H₁₃CONHCH₂CH₂CH₂, SH, SCOCH₃, SCOC₂H₅, SCOC₃H₇, SCOC₄H₉, SCOC₅H₁₁, SCOC₆H₁₃, Indolyl, Indolyl(NCOCH₃), Indolyl(NCOC₂H₅), Indolyl(NCOC₃H₇), Indolyl(NCOC₄H₉), Indolyl(NCOC₅H₁₁), Indolyl(NCOC₆H₁₃), (OH)(CH₃), (CH₃)(OCOCH₃), (CH₃)(OCOC₂H₅), (CH₃)(OCOC₃H₇), (CH₃)(OCOC₄H₉), (CH₃)(OCOC₅H₁₁), (CH₃)(OCOC₆H₁₃), PhOH, PhOCOCH₃, PhOCOC₂H₅, PhOCOC₃H₇, PhOCOC₄H₉, PhOCOC₅H₁₁, PhOCOC₆H₁₃, CONH₂, CONHCH₃, CONHC₂H₅, CONHC₃H₇, CONHC₄H₉, CONH₂C₅H₁₁, CONH₂C₆H₁₃, CH₂CONH₂, CH₂CONHCH₃, CH₂CONHC₂H₅, CH₂CONHC₃H₇, CH₂CONHC₄H₉, CH₂CONHC₅H₁₁, CH₂CONHC₆H₁₃, CH₂CH₂NHC(NH)NH₂, CH₂CH₂NHC(NH)NHC(O)CH₃, CH₂CH₂NHC(NH)NHC(O)C₂H₅, CH₂CH₂NHC(NH)NHC(O)C₃H₇, CH₂CH₂NHC(NH)NHC(O)C₄H₉, CH₂CH₂NHC(NH)NHC(O)C₅H₁₁, CH₂CH₂NHC(NH)NHC(O)C₆H₁₃, Imidazolyl, Imidazolyl(NCOCH₃), idazolyl(NCOC₂H₅), Imidazolyl(NCOC₃H₇), Imidazolyl(NCOC₄H₉), Imidazolyl(NCOC₅H₁₁), Imidazolyl(NCOC₆H₁₃), C(O)OH, C(O)OCH₃, C(O)OC₂H₅, C(O)OC₃H₇, C(O)OC₄H₈, C(O)OC₅H₁₁, C(O)OC₆H₁₃, CH₂C(O)OH, CH₂C(O)OCH₃, CH₂C(O)OC₂H₅, CH₂C(O)OC₃H₇, CH₂C(O)OC₄H₈, CH₂C(O)OC₅H₁₁, CH₂C(O)OC₆H₁₃,
R²=H, C(O)CH₃, C(O)C₂H₅, C(O)C₃H₇, C(O)C₄H₉, C(O)C₅H₁₁, C(O)C₆H₁₃
R³=H, CH₃, C₂H₅, C₃H₇, C₄H₉, C₅H₁₁, C₆H₁₃,
wherein the excipient reduces the viscosity of a high concentration biologic formulation,
wherein the biologic formulation is selected from the group consisting of a protein therapeutic, a peptide, an antibody, an antibody drug conjugate and a nucleic acid.

2. The biologic formulation of claim 1 wherein the compound is bis acetyl arginine.

3. The biologic formulation of claim 1 wherein the compound is bis acetyl lysine.

4. The biologic formulation of claim 1 wherein the compound is bis acetyl histidine.

5. The biologic formulation of claim 1 wherein the compound is bis acetyl serine.

6. The biologic formulation of claim 1 wherein the compound is bis acetyl proline.

7. The biologic formulation of claim 1 wherein the compound is bis acetyl tryptophan.

8. The biologic formulation of claim 1 wherein the compound is propionyl arginine.

9. The biologic formulation of claim 1 wherein the compound is propionyl lysine.

10. The biologic formulation of claim 1 wherein the compound is propionyl histidine.

11. The biologic formulation of claim 1 wherein the compound is propionyl serine.

12. The biologic formulation of claim 1 wherein the compound is propionyl proline.

13. The biologic formulation of claim 1 wherein the compound is propionyl tryptophan.

14. A method for reducing viscosity and/or increasing stability of a biologic formulation comprising:
combining the biologic formulation with a performance-enhancing excipient selected from the group consisting of bis acetyl arginine, bis acetyl lysine, bis acetyl histidine, bis acetyl serine, bis acetyl proline, bis acetyl tryptophan, propionyl arginine, propionyl lysine, propionyl histidine, propionyl serine, propionyl proline, propionyl tryptophan, and mixtures thereof,
wherein the biologic formulation comprises a therapeutic protein at a concentration of about 1 mg/ml to about 500 mg/ml, and a performance-enhancing excipient at a concentration of about 5 mM to about 1000 mM, to provide an enhanced formulation.

15. The method of claim 14 wherein the performance-enhancing excipient is a combination of two excipients present in the biologic formulation at about 10 wt. %:90 wt. % to about 90 wt. %: 10 wt. %.

16. The method of claim 14 wherein the performance-enhancing excipient is a mixture of propionyl serine and bis acetyl lysine in the ratio of about 10 wt. %:90 wt. % to about 90 wt. %: 10 wt. %.

17. The method of claim 14 wherein the enhanced formulation is in the form of a lyophilized powder, wherein at least one performance-enhancing excipient is present at a weight:weight concentration effective to reduce viscosity upon reconstitution with a diluent.

18. The method of claim 14 wherein the biologic formulation is selected from the group consisting of protein therapeutics, peptides, antibodies, antibody drug conjugates (ADC), nucleic acids, gene therapy and cell therapy.

19. The method of claim 14 wherein the enhanced formulation further comprises an additional excipient, wherein the additional excipient is selected from the group consisting of sugars, polyols, amino acids, amino acid derivative, surfactants, carbohydrates or combinations thereof.

20. The formulation of claim 14, further comprising a salt, a surfactant, a buffering agent, an antioxidant, an antimicrobial agent, a human serum albumin, a lipid and/or cyclodextrin.
